(19) United States
(12) Patent Application Publication
(10) Pub. No.: US 2022/0039984 A1
(43) Pub. Date: Feb. 10, 2022

(54) Orthotic Device for Treating Plantar Fasciitis

(71) Applicant: Lee A. Shirer, Willow Springs, IL (US)

(72) Inventor: Lee A. Shirer, Willow Springs, IL (US)

(21) Appl. No.: 17/444,144

(22) Filed: Jul. 30, 2021

Related U.S. Application Data
(60) Provisional application No. 63/063,273, filed on Aug. 8, 2020.

(51) Int. Cl. A61F 5/01; A61F 5/14

(52) U.S. Cl. CPC A61F 5/0111; A61F 5/14

(57) ABSTRACT

An orthotic device for treating plantar fasciitis includes a dorsal pad, an arch strap, and an Achilles strap. The arch strap releasably fastens to the dorsal pad. When the orthotic device is fitted to a human foot in an operative position, the dorsal pad covers the dorsal surface of the hind foot region and mid foot region, the arch strap is coupled to the dorsal pad with a releasable fastener and supports the plantar surface of the hind foot region and mid foot region, and the Achilles strap engages the Achilles tendon region above the heel of the foot.

This application claims the benefit of provisional U.S. Patent Application No. 63/063,273, filed Aug. 8, 2020, the entirety of which is incorporated by reference herein.

### FIELD

This application relates generally to an orthotic device for treating planer fasciitis in a human foot.

### BACKGROUND

Plantar fasciitis is a common cause of heel and foot pain, in which the plantar fascia in the human foot become injured and cause pain in the heel and/or along the sole of the foot. Although the exact causes of plantar fasciitis are not fully understood, it is generally thought to include inflammation and/or micro-tearing of the plantar fascia and/or excessive tension on the plantar fascia from a taut Achilles tendon or calf muscles. In any event, plantar fasciitis can cause extreme discomfort to the person, to the point of making it extremely painful and difficult to walk. Therefore, a simple and effective therapeutic device to relieve the pain of plantar fasciitis would be desirable.

### SUMMARY

The orthotic devices disclosed hereinafter provide a new device and method for treating plantar fasciitis.

In some arrangements, an orthotic device for treating plantar fasciitis includes a dorsal pad sized and shaped to cover the dorsal surface of a human foot, an arch strap with a releasable fastener that releasably fastens to the dorsal pad, and an Achilles strap coupled to the arch strap. The orthotic device may have an operative position configured to be fitted to a human foot in which the dorsal pad is configured to cover the dorsal surface of the hind foot region and mid foot region of the foot, the arch strap is coupled to the dorsal pad with the releasable fastener and configured to exert pressure against the plantar surface of the foot, and the Achilles strap is configured to engage the Achilles tendon region above the heel of the foot.

In some arrangements, an orthotic device for treating plantar fasciitis includes a dorsal pad and a plantar support that releasably attaches to the dorsal pad. The dorsal pad may have an interior surface, an exterior surface, and a peripheral edge forming a posterior edge and an anterior edge extending between a first lateral edge and a second lateral edge. The plantar support may include an arch strap having a posterior edge extending from a first end to a second end, a first releasable fastener disposed at the first end of the arch strap, a second releasable fastener disposed at the second end of the arch strap, and an Achilles strap having a first end attached to the posterior edge of the arch strap and a second end attached to the posterior edge of the arch strap. The first releasable fastener of the arch strap may releasably couple to the dorsal pad near the first lateral edge. The second releasable fastener of the arch strap may releasably couple to the dorsal pad near the second lateral edge.

In some arrangements, a method of treating plantar fasciitis in a foot using an orthotic device having a dorsal pad, an arch strap that releasably fastens to the dorsal pad with a releasable fastener, and an Achilles strap coupled to the arch strap is provided. The dorsal surface of the foot may be covered with the dorsal pad. The arch strap may be fastened to the dorsal pad with the releasable fastener in a selected position on the foot so as to apply pressure to the plantar surface of the foot region. The Achilles strap may be wrapped behind and in engagement against the Achilles tendon region of the foot above the heel of the foot to retain the arch strap in the selected position on the foot.

These and other aspects, arrangements, features, and/or technical effects will become apparent upon detailed inspection of the figures and the following description.

### BRIEF DESCRIPTION OF THE DRAWINGS

FIG. 1 is an isometric view of an orthotic device disassembled and in position ready to secure in an operative position about a human foot;

FIG. 2 is an isometric view of the orthic device of FIG. 1 assembled and in the operative position wrapped around a human foot;

FIG. 3 is a top (dorsal) view of the orthotic device of FIG. 1;

FIG. 4 is a bottom (plantar) view of the orthotic device of FIG. 1;

FIG. 5 is a rear (posterior) view of the orthotic device of FIG. 1;

FIG. 6 is a side (medial or lateral) view of the orthotic device of FIG. 1;

FIG. 7 is a bottom plan view of a dorsal pad of the orthic device according to a first embodiment;

FIG. 8 is a top plan view of the dorsal pad of FIG. 7;

FIG. 9 is a cross-sectional view of the dorsal pad along the line A-A in FIG. 7;

FIG. 10. is a bottom plan view of a dorsal pad of the orthotic device according to a second embodiment; and

FIG. 11 is a cross-sectional view of the dorsal pad along the line B-B in FIG. 10.

### DETAILED DESCRIPTION

Before describing the specific examples shown in the drawings, some general aspects, arrangements, and features of the disclosed orthic devices are provided. In some arrangements, an orthotic device for treating plantar fasciitis includes a dorsal pad, an arch strap, and an Achilles strap. The dorsal pad may be sized and shaped to cover the dorsal surface of the hind foot region and mid foot region of a human foot. The arch strap and the Achilles strap may, together, form a plantar support that attaches to the dorsal pad. The arch strap may releasably fasten to the dorsal pad with one or more fasteners or may be permanently attached to the dorsal pad. The Achilles strap may releasably fasten to the arch strap with one or more releasable fasteners or may be permanently attached to the arch strap. When the orthotic device is fitted to a human foot in an operative position, the dorsal pad covers the dorsal surface of the hind foot region and mid foot region, the arch strap is attached to the dorsal pad, for example with the one or more releasable fasteners, and supports the plantar surface of the hind foot region and mid foot region, and the Achilles strap wraps around the Achilles tendon region above the heel of the foot to prevent the dorsal pad and arch strap from sliding forward on the foot.

The dorsal pad may be formed of a flexible foam body. the flexible foam body may be in the form of a generally planar (although flexible out of plane to adapt to the contours of the foot) sheet of foam rubber having a relatively constant primary thickness and an outer peripheral edge in the width and length directions. The flexible foam body may have an interior side that engages the dorsal surface of the foot in the operative position. The interior side may have a primary surface extending across the interior side. The interior side may have one or more protrusions protruding outwardly from the primary surface. One or more layers of fabric may be disposed on the exterior face of the dorsal pad. The fabric may cover the all or substantially all of the exterior side of the foam body. The fabric may have a weave or other structure suitable to form a complementary portion of a hook and/or loop fastener on the arch strap so that hook-and-loop fasteners on the arch strap can fasten to the fabric.

The arch strap may be formed a strip of flexible material. The first end of the arch strap may releasably fasten to the first edge of the dorsal pad, and the second end of the arch strap may releasably fasten to the second edge of the dorsal pad. The arch strap may have a first fastener disposed at a first end thereof. The first fastener may releasably couple to a first edge of the dorsal pad on a first side of the foot when in the operative position. The arch strap may have a second fastener disposed at a second end thereof. The second fastener may releasably couple the second end of the arch strap to a second edge of the dorsal pad on a second side of the foot opposite the first side of the foot when in the operative position. In some arrangements, the second end of the arch strap may be permanently attached to a second edge of the dorsal pad on a second side of the foot opposite the first side of the foot when in the operative position, and the first end may releasably fasten to the first edge of the dorsal pad. In some arrangements, both ends of the arch strap may be permanently attached to the respective first and second lateral edges of the dorsal pad. Additional fasteners, such as two, three, four, or more, may be provided at each end of the arch strap. One or more of the fasteners may be a hook and/or loop fastener that releasably fastens to an exterior surface of the dorsal pad. The arch strap may be made of an elastic material. For example, the arch strap may be a band of elastic fabric.

One or more fastener tabs may extend from or be defined by one or both ends of the arch strap. One or more of the fasteners may be disposed on any one or more of the fastener tabs. One or more of the fastener tabs may have a hook-and-loop fastener portion on an interior side thereof. One or more of the fastener tabs may have a gripping surface with higher friction surface than the surrounding surface, such as a patch with a rough or rubbery surface, disposed on the exterior side thereof.

The Achilles strap has a first end coupled to a posterior edge of the arch strap and a second end coupled to the posterior edge of the arch strap. Either or both of ends of the Achilles strap may be permanently attached to the arch strap or may have releasable fasteners, such as hook and loop fasteners, that can releasably attach to the arch strap. The Achilles strap may be made of an elastic material. For example, the Achilles strap may be a band of elastic fabric.

These and other features and aspects of the orthotic device will become apparent upon inspection of the drawings in view of the description thereof hereinafter.

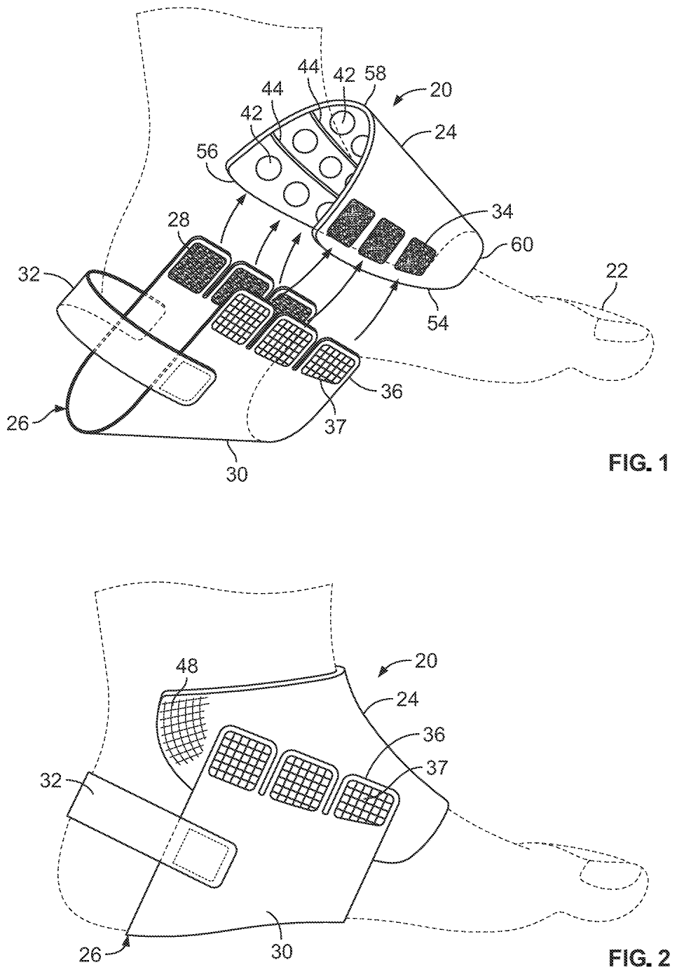

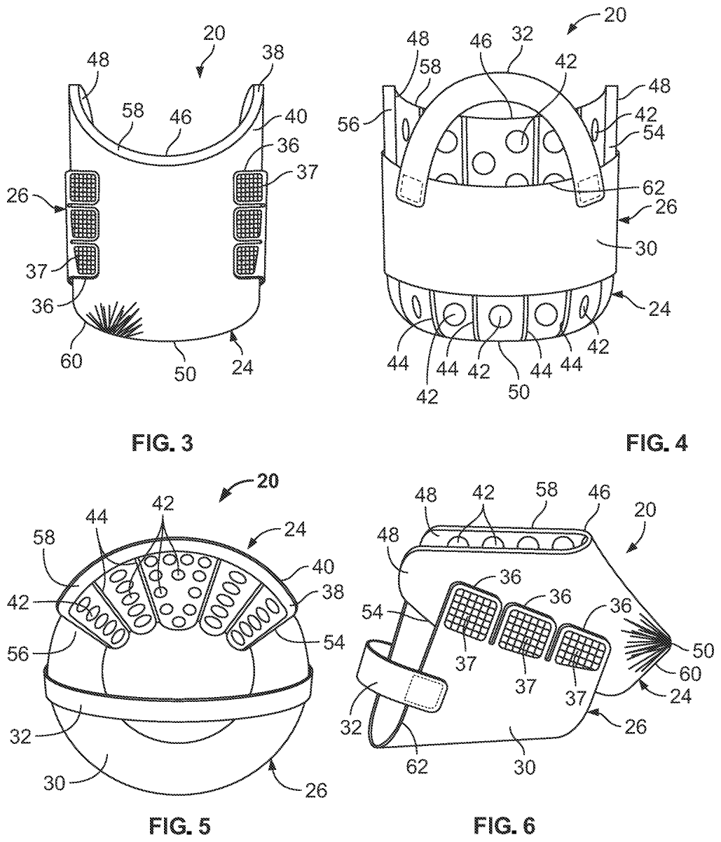

Turning now to the exemplary arrangements of the drawings, FIGS. 1 and 2 show an orthotic device 20 adapted to treat plantar fasciitis on a human foot 22 with the foot shown for ease of visualization, and FIGS. 3-6 show the orthotic device 20 from different angles without the foot present. The orthotic device 20 includes a dorsal pad 24 and a plantar support 26. The plantar support 26 includes an arch strap 30 and an Achilles strap 32. The plantar support 26 releasably attaches to the dorsal pad 24 with fasteners 28, such that the plantar support 26 can be repeatedly completely removed from the dorsal pad 24 or securely attached to the dorsal pad 24, as desired by the user. As best seen in FIG. 1, when the plantar support 26 is disengaged from the dorsal pad 24, the orthotic device 20 can be more easily put on the foot 22 and taken off of the foot. As best seen in FIG. 2, when the orthotic device 20 is in its operative position on the foot 22 to relieve plantar fasciitis in the foot, the plantar support 26 is fastened to the dorsal pad 24, the dorsal pad 24 wraps over and lies against the dorsal portion of the hind- and mid-foot regions, the arch strap 30 wraps around the hind- and mid-foot regions and presses against the plantar surface of the heel and sole of the foot, and the Achilles strap 32 wraps snuggly around and against the posterior of the ankle against Achilles tendon area just above heel. In the operative position, the arch strap 30 applies pressure against the sole (plantar) portion of the mid-foot and optionally against the hind-foot, for example, against the arch of the foot and/or the heel. The dorsal pad 24 wraps over the top (dorsal) portion of the foot and holds the arch strap 30 in place against the sole of the foot. The Achilles strap 32 wraps around the posterior side of the ankle in the region of the Achilles tendon just above the heel to prevent the arch strap 30 and the dorsal pad 24 from sliding forward on the foot. Thus, in the operative position, the dorsal pad 24, arch strap 30, and Achilles strap 32 work together to maintain the orthotic device 20 in the desired position on the foot so that the arch strap 30 provides steady pressure against the sole of the foot, and thereby also against the plantar fascia, which may help relieve pain and/or treat plantar fasciitis.

Focusing now more particularly on the various individual structures of the orthotic device 20, the dorsal pad 24 is a flexible member adapted to wrap around the top of the foot and to releasably attach with the plantar support 26 as described in detail hereinafter. The dorsal pad 24 is a relatively thin sheet member that can flex to conform to the outer contours of the dorsal region of the foot. The dorsal pad 24 has a generally V-shaped outer periphery that forms a shield shape that is symmetric about a central axis A. The lateral edges of the dorsal pad 24 are angled relative to the central axis A such that the dorsal pad 24 is wider at the posterior edge 58 and narrower at the anterior edge 60. The posterior edge 58 forms a notch 46 on the central axis A and two wings 48 on opposite sides of the central axis A. The notch 46 is designed to engage against and receive the anterior apex of the ankle, and the two wings 48 wrap at least partly around the opposite left and right sides of the ankle. The anterior edge 60 is generally parallel to the posterior edge 58, thereby forming an apex 50 also on the central axis A. As can best be seen in FIGS. 1 and 2, the V-shaped outer periphery allows the dorsal pad 24 to fit more closely to the contour of the dorsal region of the foot 22 and around the ankle and provide a long surface along each side of the foot along which the arch strap 30 can attach to the dorsal pad 24. Preferably, the dorsal pad 24 is formed of a material that is stiffer than the arch strap 30 in order to spread out the pressure against the dorsal surface of the foot to prevent creating a tourniquet effect along the dorsal surface. For example, the dorsal pad 24 may be formed with a thick leather pad or thick foam rubber pad or other similarly flexible and sturdy material. Also, the interior surface of the dorsal pad 24 may be soft and pliable enough to conform at least partially to the contours of the dorsal surface of the foot in order to further ensure even distribution of pressure against a large surface area of the dorsal surface.

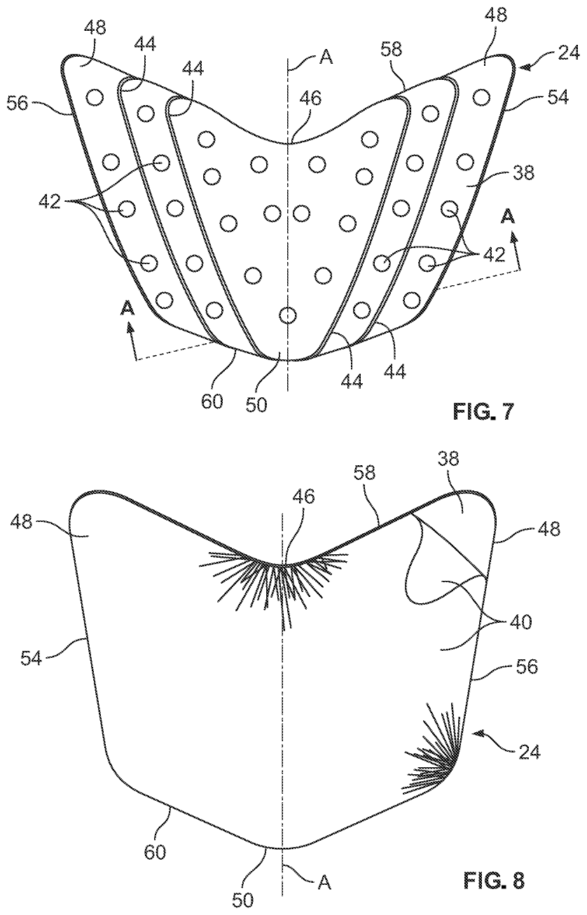

As best seen in FIGS. 7-9, the dorsal pad 24 has a foam pad 38 forming a base layer and a layer of fabric 40 covering the exterior face (top surface) of the foam pad 38. The fabric 40 is preferably cut to match the outer peripheral shape of the foam pad 38. The fabric 40 may be laminated to the top surface of the foam pad 38, for example, with adhesive or heat. FIG. 8 shows a small portion of the fabric 40 pulled away from the top surface of the foam pad 38 for illustration, it being understood that the fabric 40 is preferably secured along its entire area to the top surface of the foam pad 38. In this arrangement, the fabric 40 is a loop fastener fabric that forms the loop portion of a hook-and-loop fastener so that complementary hook portions of the hook-and-loop fastener carried by the arch strap 30 can be fastened to the dorsal pad 24 at any desired location across the entire exterior surface of the dorsal pad 24. This allows the user to adjust the attachment points of the arch strap to the dorsal pad to a much wider range of locations, which enables to the user to select a wide range of pressures against the plantar surface. It also allows the user to easily adjust the amount of pressure at different areas of the plantar surface. For example, the arch strap could be attached so that a higher pressure is applied against the heel region and a lower pressure is applied against the arch region, vice versa, or so that even pressure is applied against both the heel region and the arch region. The foam pad 38 may be formed with any suitable foam rubber material. Preferably the foam pad 38 is made of a closed-cell foam, such as a neoprene foam, gym rubber foam, or a polyethylene foam. In some arrangements, the foam pad 38 is made of a cross-linked, closed-cell, heat moldable, polyethylene foam, such as a medium density Volara foam or a medium density AliPlast™ foam by AliMed, Inc. of Dedham, Mass. The foam pad 38 may be formed by any appropriate process, for example by vacuum forming or molding. Preferably, the foam pad 38 has the form of a sheet with a generally uniform thickness (other than the protrusions and/or grooves described hereinafter) between about 1 mm and 25 mm, more preferably between about 2 mm and 12 mm, and most preferably between about 3 mm and 7 mm, although other thicknesses may be used.

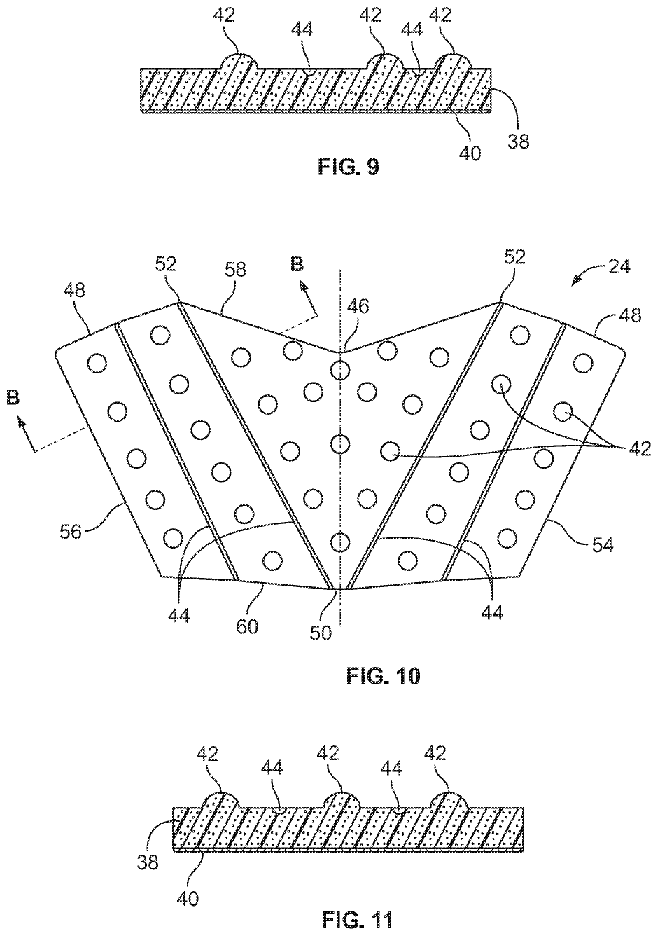

As best seen in FIGS. 7 and 9, several protrusions 42 are disposed on the interior side (bottom surface) of the foam of the foam pad 38. The protrusions 42 may help prevent a tourniquet effect and maintain healthy blood-flow to the foot when the orthotic device 20 is in the operative position wrapped around a user's foot 22. The protrusions 42 are preferably integral parts of the foam pad 38 that are formed, such as during the molding process, directly on the interior face of the foam pad. The protrusions 42 protrude outwardly from the primary surface of the interior face. Preferably, the protrusions 42 are symmetric about the central axis A. In some arrangements, the protrusions 42 may optionally be omitted, such that the interior face of the foam pad 38 is substantially smooth.

The protrusions 42 are shaped as portions of spheres projecting outwardly from the interior surface of the foam pad 38; however, the protrusions may have other shapes as well. Preferably, the protrusions 42 protrude between about 1 mm and 5 mm out from the primary surface of the interior face, although the protrusions may protrude further from the primary surface. The protrusions are spaced apart from each other and disposed over the entire interior surface of the dorsal pad 24. In this arrangement, the protrusions are arranged in an array such that there are adjacent lines of protrusions parallel to and offset from the left and right lateral edges 56, 54 of the dorsal pad 24. In this array, a first line of the protrusions 42 extends at an axial angle spaced inwardly from and adjacent to each lateral side, a second line of protrusions 42 extends at an axial angle spaced inwardly from and adjacent to the first line of protrusions on each lateral side, a third line of protrusions 42 extends along the medial side of and spaced inwardly from the second line of protrusions on each lateral side, and additional protrusions are located near the ankle notch 46. The spacing and arrangement of the protrusions 42 is preferably selected to reduce or minimize any blockage of blood flow, i.e., a tourniquet effect, along the dorsal surface of the foot. The array of the protrusions 42 shown in this exemplary arrangement is also believed to provide the wearer maximum comfort. However, other arrays of the protrusions 42 may be used.

Pre-defined sizing markings, such as grooves 44 disposed on the interior side (bottom surface) of the foam of the foam pad 38, may be provided to help delineate where to trim the pad when customizing the size of the dorsal pad 24 for the foot size of an individual wearer. However, the grooves 44 do not form a part of the present invention.

The arch strap 30 is made of a flexible fabric material to be able to easily mold itself to the contours of the plantar surface, i.e., the sole, of the foot under the arch and heel. Preferably, the arch strap 30 is made of an elastic material, such as an elastic band and/or elastic fabric, in order to apply pressure against the plantar surface of the foot in the operative position. The arch strap 30 is in the form of a strip or band of flexible fabric extending lengthwise from a first end to a second end. The strip or band has a width selected to cover a portion of a human foot from about the mid-foot or arch region to the hind-foot or heel region. In some arrangements, the strip or band has a length between about 50 mm and about 250 mm, and more preferably between about 100 mm and about 200 mm. In some arrangements, the band has a width between about 25 mm and about 200 mm, and more preferably between about 75 mm and about 100 mm. However, the strip or band may have other length and width dimensions. Making the arch strap wide enough to press against both the arch (mid-foot region) and the heel (hind-foot region) of the plantar surface provides improved relief over a device that only covers the arch region of the foot. This is counterintuitive from previously known therapies, which tend to focus primarily on only the arch (mid-foot) region of the plantar surface.

One or more fasteners 28 are disposed at each of the opposite ends of the arch strap 30. The fasteners 28 are positioned to engage with complementary fasteners or fastener regions 34 disposed on the exterior surface of the dorsal pad 24. In this exemplary arrangement, three fasteners 28 are disposed on each opposite end of the arch strap 30. The fasteners 28 are disposed on the interior face of the arch strap 30 so as to engage against the fastener regions 34 on the exterior face along the opposite side edges of the dorsal pad 24. The fastener regions 34 may be formed of specific fasteners, such as a loop portion of a hook-and-loop fastener pair, or may be formed of a larger region of material suited for fastening with the fasteners 28, such as the surface of a fabric covering that can attach to the hook portion of a hook-and-loop fastener pair. The fasteners 28 are shown as hook and loop fasteners portions that can releasably attach to the exterior face of the dorsal pad 24 at the fastener regions 24, such as to the layer of fabric 40, or to complementary portions of hook-and-loop fasteners attached to the dorsal pad. However, other types of fasteners 28, such as snaps, hooks, clasps, and so on could be used, as well as more or fewer of the fasteners.

Preferably, two or more separated fastener tabs 36 are disposed at each opposite end of the arch strap 30. Each fastener tab 36 is separated from the adjacent fastener tab or tabs 36, for example by slit or space extending inwardly from the respective end of the arch strap or by a tab that is attached to and extends from the end of the arch strap. In the present arrangement, there are three separate fastener tabs 36 disposed along each of the two ends of the arch strap 30, and the hook portion of a hook-and-loop fastener 28 is disposed on the interior surface of each fastener tab 36. In some arrangements, only two fastener tabs 36 are disposed at each end of the arch strap 30. However, more or fewer fastener tabs 36 may be formed at either end of the arch strap 30. The separate fastener tabs 36 separated by slits or spaces may in some instances make it easier for the user to fasten and unfasten the fasteners 28 to the fasteners 34 and/or adjust the fit of the orthotic device 20 to the foot. However, in other arrangements, the separate fastener tabs 36 may be omitted. In some arrangements, each fastener tab 36 is formed of a strip of the hook portion of a hook-and-loop fastener, and is preferably permanently attached to the respective end of the arch strap, for example by stitching, adhesive, or other permanent fastening mechanism. The fastener tabs 36 make it easier to attach the arch strap to the dorsal pad in more varied positions, thereby making it easier to adjust the pressure that the arch strap provides against the sole of the foot. The plurality of smaller fastener tabs 36 also are easier to unfasten from the dorsal pad than a single large fastener.

A grip surface 37 optionally may be disposed on the exterior side of each of one or more of the fastener tabs 36. The grip surface can be any surface that provides higher friction or traction to a user's fingers than the surrounding portion of the tabs or arch strap to help the user grip and manipulate the tabs. For example, the grip surface 37 may have a rough surface or a rubbery surface. In other examples, however, where each fastener tab 36 is defined by a hook fastener (of a hook-and-loop fastener 28) that is attached to the respective end of the arch strap 30, no special grip surface 37 is disposed on the back side of the hook fastener strip. The grip surface 37 may help the user more easily fasten or unfasten the fasteners 28 to or from the dorsal pad 24.

The Achilles strap 32 forms a loop around the posterior surface of the ankle above the heel to prevent the orthotic device 20 from sliding forward along the foot in the operative position, thereby maintaining the orthotic device 20 in a preferred position around the foot. The Achilles strap 32 preferably is made of a flexible fabric, and more preferably an elastic material such as an elastic band or elastic fabric for similar reasons as with the arch strap 30. Each of the opposite ends of the Achilles strap 32 is attached to a posterior edge 62 of the arch strap 30 on opposing legs of a U-shape formed by the arch strap 30. The ends of the Achilles strap 32 may be permanently attached to the arch strap 30 with stitching, adhesive, or other securing mechanism sufficient to permanently attach the Achilles strap 32 to the arch strap 30. In some arrangements, one or both of the ends of the Achilles strap may be releasably fastenable to the arch strap, for example with hook-and-loop fasteners, snaps, or other fasteners. By preventing the arch strap from slipping forward on the foot, the Achilles strap helps ensure that the arch strap provides the selected pressure against the heel portion of the plantar surface, which can help ensure more effective pain relief from the plantar fasciitis.

Turning now to FIGS. 10 and 11, a second exemplary configuration of the dorsal pad 24 is shown. In this example, the dorsal pad 24 has a slightly different, more angular outer peripheral shape, and the array of the protrusions 42 near the ankle notch 46 is slightly different. Of primary difference in this example, the posterior edge 58 of the dorsal pad 24 has an M-shape rather than a simple V-shape, and the grooves 44 are aligned slightly differently. However, in all other aspects, the dorsal pad shown in FIGS. 10 and 11 is substantially the same as the dorsal pad 24 shown in FIGS. 7-9, and the numbering of the various features and accompanying description already provided above is not repeated here for brevity, the reader being directed to the prior descriptions thereof.

Turning again to FIGS. 1 and 2, to secure the orthotic device 20 in its operative position on a foot, the dorsal pad 24 is wrapped around and against the dorsal side of the hind- and mid-foot region with the interior surface engaged against the foot (or a sock covering the foot), the ankle notch 46 against the anterior surface of the ankle, and the apex 50 pointing toward the toes. The plantar support 26 is fastened to the dorsal pad 24 with the arch strap 30 wrapped around and against the sole of the foot, preferably in the hind- and mid-foot region, and the Achilles strap 32 wrapped around and against the Achilles tendon region directly above the heel. One end of the arch strap 30 is fastened to the lateral edge of the dorsal pad 24, and the other end of the arch strap 30 is fastened to the medial edge of the dorsal pad 24. Specifically, the fasteners 28 on the one end of the arch strap 30 are secured to the cooperating fastener regions 34 along the right lateral edge 54 of the dorsal pad 24, and the fasteners 28 on the other end of the arch strap 30 are secured to the cooperating fastener regions 34 along the left lateral edge 56 of the dorsal pad 24. In this example, the hook portion of the hook-and-loop fasteners 28 on each fastener tab 36 is secured to the fastener regions 34 of the layer of fabric 40 on the exterior surface of the dorsal pad 24. The pressure exerted by the arch strap 30 against the plantar fascia can be easily adjusted by adjusting where the hook fasteners 28 on the fastener tabs 36 are engaged against the fabric 40 on the dorsal pad 24. Thus, with the use of hook-and-loop fasteners 28 and a fastening region 34 formed by the fabric 40 extending across the entire area or a large portion of the area of the exterior surface of the dorsal pad, the orthotic device 20 can be adjusted easily to fit a wide range of different sizes of foot and to provide a wide range of pressure for each foot, depending on where the user attaches the fastener tabs 36 to the dorsal pad 24.

An orthotic device according to some aspects disclosed herein provides improved relief for plantar fasciitis by applying more pressure against swollen cells of the fascia. It is believed that this provides improved relief by pressing excess fluid from the swollen cells, thereby reducing pain when walking. Using a strong elastic band for the arch strap can provide more pressure against the plantar surface, thereby squeezing more excess fluid from the inflamed cells, and providing improved pain relief. Providing the hook-and-loop fasteners as described herein allows the arch strap to be fastened to the dorsal pad at essentially any position across the exterior surface of the dorsal pad which provides for near infinite adjustability of the arch strap, thereby allowing the user to adjust the amount and position of pressure against a wide area of the plantar surface, including both the mid-foot region (arch) and the hind-foot region (heel). Providing the arch strap fully detachable from the dorsal pad allows the user to adjust the fit and pressure of the arch strap more fully than if the two structures were to be permanently attached to each other. In addition, the orthotic device can be adjusted to fit to a wide range of sizes and shapes of foot, for example, by trimming the size of the dorsal pad with scissors and/or by adjusting the attachment locations of the arch strap to the dorsal pad. By using a dorsal pad that has a stiffer structural profile, for example using the foam rubber as a base, rather than simply using another piece of flexible fabric or an elastic band, the pressure against the top foot may be dispersed more evenly across the dorsal surface so as to reduce or prevent unwanted constriction of the blood vessels in the dorsal surface of the foot. This helps apply the pressure where most needed against the dorsal surface of the foot without applying a full tourniquet around the foot, thereby allowing the user to apply greater pressure to the affected plantar fascia without cutting off all blood supply to the toes. Of course, some arrangements may implement fewer of the advantages or any of various combinations of these features and/or advantages.

The orthotic devices disclosed herein can be worn comfortably while sleeping without stopping the flow of blood to the distal end of the foot. When so worn in the operative position, the inventor has found that the orthotic devices significantly relieve the pain of walking caused by plantar fasciitis when first arising from bed. Often, a shoe can be worn around the orthotic device during the day, for example, which the inventor has found also can relieve the pain from plantar fasciitis after sitting for long periods. Thus, the orthotic devices disclosed herein provide a simple and effective method of treating plantar fasciitis that is easily adaptable to a wide range of foot sizes and shapes, as well as a wide range of severity and location of plantar fasciitis.

This detailed description is to be construed as examples only and does not describe every possible embodiment, as describing every possible embodiment would be impractical, if not impossible. One could implement numerous alternate embodiments, using either current technology or technology developed after the filing date of this application. Thus, while specific exemplary forms are illustrated and described herein, it is to be understood that any of the various aspects, arrangements, and/or features disclosed herein may be combined with any one or more of the other aspects, arrangements, and/or features disclosed herein in a manner that would be understood by a person of ordinary skill in view of the teachings of this disclosure.

I claim:

1. An orthotic device for treating plantar fasciitis, the orthotic device comprising:
a dorsal pad sized and shaped to cover the dorsal surface of a human foot;
an arch strap with a releasable fastener that releasably fastens to the dorsal pad; and
an Achilles strap coupled to the arch strap;
wherein the orthotic device has an operative position configured to be fitted to a human foot in which the dorsal pad is configured to cover the dorsal surface of the hind foot region and mid foot region of the foot, the arch strap is coupled to the dorsal pad with the releasable fastener and configured to exert pressure against the plantar surface of the foot, and the Achilles strap is configured to engage the Achilles tendon region above the heel of the foot.

2. The orthotic device of claim 1, wherein the dorsal pad comprises a flexible foam body.

3. The orthotic device of claim 2, wherein the flexible foam body comprises:
an interior side for engaging the dorsal surface of the foot in the operative position, wherein the interior side comprises a primary surface extending across the interior side and a plurality of protrusions protruding outwardly from the primary surface.

4. The orthotic device of claim 1, wherein the arch strap comprises:
a strip of flexible material extending from a first end to a second end; and
a first fastener disposed at the first end;
wherein the first fastener releasably couples to a first lateral edge of the dorsal pad.

5. The orthotic device of claim 4, wherein the arch strap further comprises:
a second fastener disposed at the second end;
wherein the second fastener releasably couples to a second lateral edge of the dorsal pad opposite the first lateral edge.

6. The orthotic device of claim 5, wherein at least one of the first fastener and the second fastener comprises a hook and loop fastener portion that releasably fastens to an exterior surface of the dorsal pad.

7. The orthotic device of claim 4, wherein the arch strap further comprises a fastener tab, and wherein the fastener tab carries the fastener.

8. The orthotic device of claim 4, wherein the arch strap comprises an elastic material.

9. The orthotic device of claim 1, wherein a fabric is disposed on an exterior face of the dorsal pad, wherein the fabric forms a complementary portion of a hook and loop fastener portion on the arch strap.

10. The orthotic device of claim 1, wherein the Achilles strap comprises a first end coupled to a posterior edge of the arch strap and a second end coupled to the posterior edge of the arch strap.

11. The orthotic device of claim 10, wherein each of the first and second ends of the Achilles strap is permanently attached to the arch strap.

12. An orthotic device for treating plantar fasciitis, the orthotic device comprising:
a dorsal pad having an interior surface, an exterior surface, and a peripheral edge forming a posterior edge and an anterior edge extending between a first lateral edge and a second lateral edge; and
a plantar support that releasably attaches to the dorsal pad, the plantar support comprising:
an arch strap having a posterior edge extending from a first end to a second end;
a first releasable fastener disposed at the first end of the arch strap;
a second releasable fastener disposed at the second end of the arch strap; and
an Achilles strap having a first end attached to a posterior edge of the arch strap and a second end attached to the posterior edge of the arch strap;
wherein the first releasable fastener of the arch strap releasably couples to the dorsal pad near the first lateral edge and the second releasable fastener of the arch strap releasably couples to the dorsal pad near the second lateral edge.

13. The orthotic device of claim 12, wherein the dorsal pad comprises a flexible sheet of foam rubber.

14. The orthotic device of claim 12, wherein the arch strap comprises a flexible elastic band.

15. The orthotic device of claim 12, wherein the dorsal pad comprises a plurality of protrusions disposed on the interior surface.

16. The orthotic device of claim 12, wherein the dorsal pad comprises a notch along the posterior edge, and wherein each of the first lateral edge and the second lateral edge is angled relative to a central axis of the dorsal pad such that the dorsal pad is wider at the posterior edge than at the anterior edge.

17. The orthotic device of claim 12, wherein the dorsal pad comprises a fabric at least partly covering the exterior surface, wherein each of the first and second fasteners comprises hook and loop fastener portions that releasably fasten to the fabric at a plurality of locations across the exterior surface.

18. A method of treating plantar fasciitis in a foot using an orthotic device comprising a dorsal pad, an arch strap that releasably fastens to the dorsal pad with a releasable fastener, and an Achilles strap coupled to the arch strap, the method comprising the steps:
covering the dorsal surface of the foot with the dorsal pad;
fastening the arch strap to the dorsal pad with the releasable fastener in a selected position on the foot so as to apply pressure to the plantar surface of the foot region, and
wrapping the Achilles strap behind and in engagement against the Achilles tendon region of the foot above the heel of the foot to retain the arch strap in the selected position on the foot.
